(19) United States
(12) Patent Application Publication
(10) Pub. No.: US 2009/0322630 A1
(43) Pub. Date: Dec. 31, 2009

(54) BLOOD GLUCOSE LEVEL MEASUREMENT AND WIRELESS TRANSMISSION UNIT

(75) Inventors: Alf Friman, Vaxjo (SE); Ulrich Kraft, Hofheim (DE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: LifeScan Scotland Ltd., Iverness (UK)

(21) Appl. No.: 12/301,753

(22) PCT Filed: May 22, 2006
(86) PCT No.: PCT/EP2006/004852

§ 371 Date: Apr. 27, 2009

(51) Int. Cl. H01Q 1/00; G01N 33/48; H01Q 1/38

(52) U.S. Cl. 343/720; 73/61.43; 343/700 MS

(57) ABSTRACT

Described and illustrated herein are various embodiments of a blood glucose level measuring unit provided with wireless communication which both is small in dimensions and has radiation characteristics which substantially direction independent especially at small distances.

### CROSS-REFERENCE

This application claims priority from International Application number PCT/EP2006/004852, filed May 22, 2006, to which applicant claims benefits of priority and which is fully incorporated herein by reference.

The present invention relates to a blood glucose level measuring unit comprising a housing and an antenna and being adapted for wireless data transmission to further devices of a blood glucose system.

Diabetes mellitus is a chronic metabolic disorder caused by an inability of the pancreas to produce sufficient amounts of the hormone insulin so that the metabolism is unable to provide for the proper absorption of sugar and starch. This failure leads to hyperglycemia, i.e. the presence of an excessive amount of glucose within the blood plasma. Persistent hyperglycemia causes a variety of serious symptoms and life threatening long term complications such as dehydration, ketoacidosis, diabetic coma, cardiovascular diseases, chronic renal failure, retinal damage and nerve damages with the risk of amputation of extremities. Because healing is not yet possible, a permanent therapy is necessary which provides constant glycemic control in order to always maintain the level of blood glucose within normal limits. Such glycemic control is achieved by regularly supplying external insulin to the body of the patient to thereby reduce the elevated levels of blood glucose.

External insulin was commonly administered by means of typically one or two injections of a mixture of rapid and intermediate acting insulin per day via a hypodermic syringe. While this treatment does not require the frequent estimation of blood glucose, it has been found that the degree of glycemic control achievable in this way is suboptimal because the delivery is unlike physiological insulin production, according to which insulin enters the bloodstream at a lower rate and over a more extended period of time. Improved glycemic control may be achieved by the so-called intensive insulinotherapy which is based on multiple daily injections, including one or two injections per day of long acting insulin for providing basal insulin and additional injections of rapidly acting insulin before each meal in an amount proportional to the size of the meal. Although traditional syringes have at least partly been replaced by insulin pens, the frequent injections are nevertheless very inconvenient for the patient.

Substantial improvements in diabetes therapy have been achieved by the development of blood glucose systems relieving the patient of the daily use of syringes or insulin pens. Such blood glucose systems usually comprise a battery-operated insulin pump and a separate battery-operated control unit. The insulin pump allows for the delivery of insulin in a more physiological manner and can be controlled to follow standard or individually modified protocols to give the patient a better glycemic control over the course of a day. It can be constructed as an implantable device for subcutaneous arrangement or can be constructed as an external device that is carried on the body of the patient.

The operation of the insulin pump can be controlled and modified by means of the control unit. For example, delivery of suitable amounts of insulin by the insulin pump requires that the patient frequently determines his or her blood glucose level and inputs this value into the control unit, which then calculates a suitable modification to the default or currently in use insulin delivery protocol, i.e. dosage and timing, and subsequently communicates with the insulin pump to adjust its operation accordingly. The determination of blood glucose concentration is performed by means of a suitable battery-operated measuring device such as a hand-held electronic meter which receive blood samples via enzyme-based test strips and calculates the blood glucose value based on the enzymatic reaction. Advantageously, the measuring device is an integral part of the blood glucose system, so that the measured value is automatically delivered to the control unit. In this regard, the measuring device may be integrated into the housing of the control unit or may be provided as a separate device communicating with the control unit. Further, it may be necessary to use the control unit each time the patient eats to instruct the pump to administer a specified amount of insulin to cover that meal. Recently, a more or less closed-loop control has been realized in which the control unit modifies the insulin delivery protocol automatically.

In view of the permanence of the therapy, it is desirable to provide the diabetic patient with flexibility, convenience and ease of use in order to increase the quality of his or her life. In this regard, it is evident that cable connections between the individual devices of a blood glucose system are disadvantageous. Thus, it is known to provide a wireless communication link. For example, WO 2005/041432 discloses to use a cellular phone being provided with glucose level measuring means which phone transmits data to an insulin pump via an infrared link and to a control unit via a wireless network.

With respect to the wireless connection between the insulin dispenser on the one hand and the control unit and/or the measuring unit on the other hand, it is desirable that this connection is sufficiently stable and does not depend on the position of the devices relative to each other. Furthermore, the quality of the emitted/received signal should not be influenced by the amount of tissue which is disposed between the emitter and the receiver. For example, the transmission quality should be independent from the fact that the measuring unit might be surrounded by the user's hand. In particular, in this special application of a blood glucose system the distance between the dispenser unit on the one hand and the control unit and/or measuring unit is comparatively small with the result that the coverage of the antenna should be good especially at smaller distances.

Furthermore, since the measuring and/or control unit usually have to be carried permanently by a patient, it should be small in dimensions and should have a geometry that has no protrusions or the like. Especially, this requires the antenna to be small to enable including it into the design of the housing of the unit.

Therefore, it is the object of the present invention to provide a blood glucose measuring unit adapted for wireless communication which both is small in dimensions and has radiation characteristics which are almost direction independent especially at small distances.

This object is achieved by a blood glucose level measuring unit comprising a housing and an antenna and being adapted for wireless data transmission to further devices of a blood glucose system, characterized in that the antenna is disposed within the housing and comprises:
a dielectric core element having a first end and a second end and defining a surface extending between the first end and the second end, and a conductor with:
a longitudinal section extending on the surface between the first end and the second end,
a first circumferential section which is connected to the end of the longitudinal section closest to the first end and extends transversely to the longitudinal section along the surface,
a second circumferential section which is connected to the longitudinal section spaced from the end closest to the second end and extends transversely to the longitudinal section along the surface,
a first connector connected to the end of the longitudinal section closest to the second end, and
a second connector connected to the second circumferential section.

The measuring unit according to the present invention shows on the one hand homogeneous transmission characteristics which means that the radiation efficiency is almost direction independent. Furthermore, the sensitivity of the antenna is not influenced by the relative position of the antenna with respect to the emitter. Due to the design of the antenna having the circumferential sections, the actual dimensions are reduced while the required electrical length is still the same.

In a preferred embodiment, the core element has two surface portions which are inclined with respect to each other. Preferably, the angle formed between the portions is within the range between 90° and 135° and, more preferably of 130°. The longitudinal section extends along the first portion while the circumferential sections extend both along the first and the second portion. This leads to a curvature of the circumferential sections which results in a further improvement of the transmission characteristics. In particular, by placing the elements of the antenna in different planes the risk of lost transmission coverage in case a certain plane is blocked can be reduced. Furthermore, the total antenna volume formed by the dielectric core is increased which results in a better antenna performance.

Furthermore, it is preferred that the connectors are provided with intermediate sections, the free end of which are connected to spring sections. The spring sections allow for an easy bonding of the antenna with the core element on a printed circuit board (PCB). Since the spring sections are biased in direction of the PCB, when the antenna is mounted on it with the lower surface abutting the board, no further soldering joints are required. This facilitates assembly of the measuring unit and reduces the production costs.

In the following, a preferred embodiment of the present invention is described with respect the drawings in which

FIG. 1 shows a blood glucose system including a measuring unit according to the present invention,

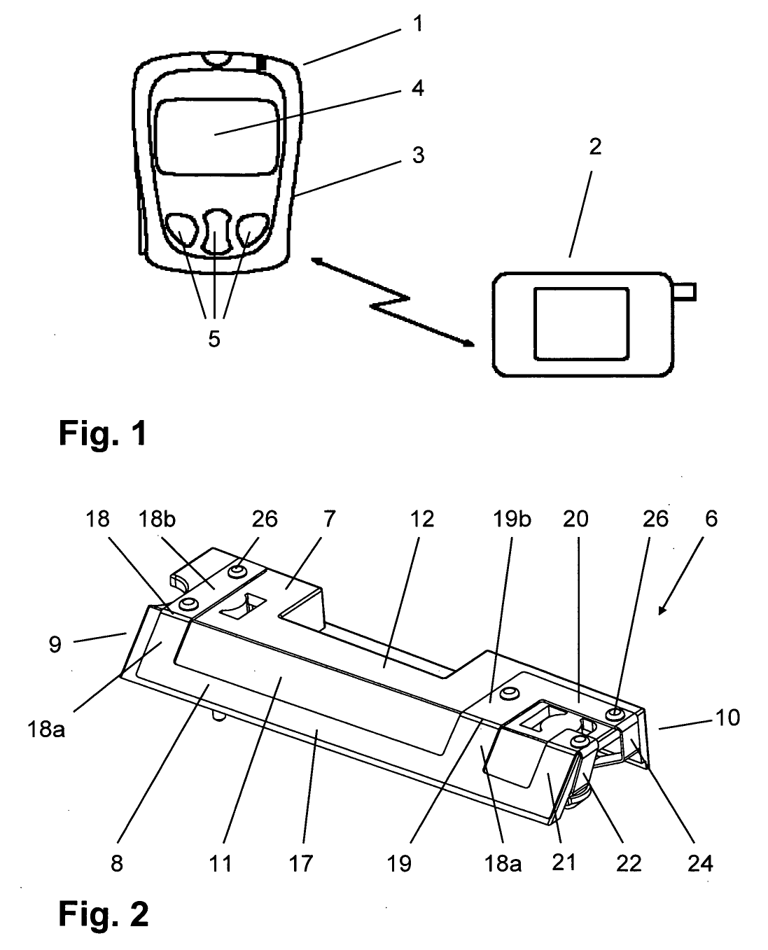

FIG. 2 shows an embodiment of an antenna of a measuring unit according to the present invention in top view,

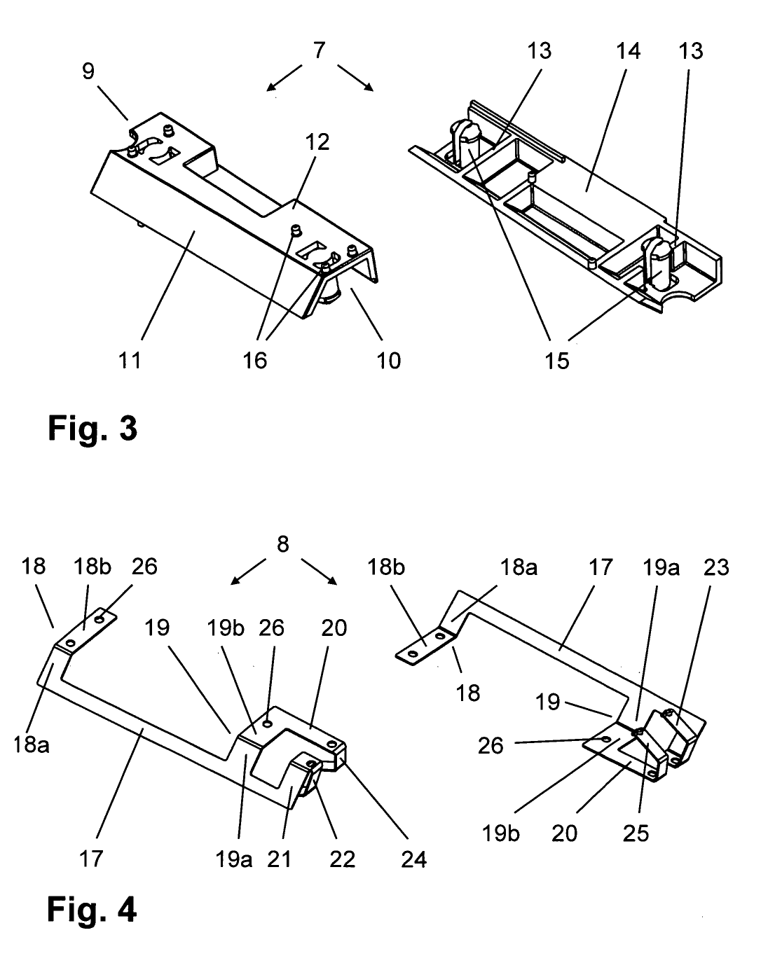

FIG. 3 shows the core element of the antenna of FIG. 2 in top and bottom view, and

FIG. 4 shows the antenna conductor of the antenna of FIG. 2 in top and bottom view.

In FIG. 1, a blood glucose system is schematically shown comprising a measuring and control device 1 and an insulin pump 2 for dispensing insulin to the blood circuit of a patient. In this preferred embodiment, the blood glucose measuring unit and the control unit are combined in a single device 1 having a common housing 3. However, in general the measuring unit on the one hand and the control unit on the other hand can be separate devices. Furthermore, a display 4 and control buttons 5 are integrated in the housing 3 of the device 1 which allow for adjusting it with regard to the patient's parameters like height, weight etc.

The measuring and control device 1 is adapted for wireless communication with other components of the blood glucose system which is in this case only the insulin pump 2. Therefore, device 1 is provided with a radio frequency (RF) transceiver (not shown) and an antenna 6 described in detail below. Both the antenna 6 and the transceiver are disposed within the housing 3. The insulin pump 2 may either be designed to be disposed subcutaneously on a patient or may be a device which is carried by the patient in a usual manner.

The antenna 6 of the measuring unit which, in this preferred embodiment, is a part of device 1 is connected to the RF-transceiver and is shown in detail in FIG. 2. The antenna 6 comprises a dielectric core element 7, which is shown separately in FIG. 3, and a conductor 8 shown in FIG. 4, wherein the conductor 8 is arranged on the surface of the core element 7.

As can be seen in FIG. 2 and 3, the core element 7 has a first end 9 and a second end 10 wherein a surface of the core element 7 is defined between the ends 9, 10. In this preferred embodiment the surface comprises a first portion 11 and a second portion 12. The portions 11, 12 are inclined with respect to each other to define an angle of about 130°. However, while in this preferred embodiment the angle is chosen to be 130° it can generally be within the range between 90° and 135°. Furthermore, the core element 7 has a lower surface which is defined by bars 13 and a face 14 which run spaced from and parallel to the second portion 12 of the surface.

Further, the core element 7 is provided with rods 15 mounted on the lower side of the second portion 12 which rods extend beyond the lower surface. The rods 15 are provided with resilient protrusions which extend perpendicular with respect to the rods 15 and which can be bent inwardly. Thus, the rods 15 are effective as snap elements which allow for securing the core element 7 to a circuit board (not shown) being part of the device 1.

The surface of the core element 7 comprises a plurality of fixing pins 16 arranged on the second portion 12. The pins 16 can protrude into openings provided in the conductor 8 to fix the latter in a predetermined position on the surface of the core element 7.

In FIG. 4, the conductor 8 is shown both in top and bottom view. In the assembled state shown in FIG. 2, the conductor 8 will be mounted on the surface of the core element 7 comprising first and second portions 11, 12. The conductor 8 has a longitudinal section 17 which extends on the first portion 11 between the first end 9 and the second end 10 along the length of the core element 7. At the end of the longitudinal section 17 which is closest to the first end 9, a first circumferential section 18 is provided which is connected to the longitudinal section 17 extending generally transversely with respect to it. In this preferred embodiment, the first circumferential section 18 is arranged perpendicular to the longitudinal section 17 and disposed both on the first portion 11 and the second portion 12 being bent suitably to follow the shape of the core element 7. A first part 18a of the first circumferential portion 18 is arranged on the first section 11 and a second part 18b on the second section 12. The angle the first and the second part 18a, 18b form with respect to each other corresponds to the angle between the first and the second portion 11, 12 of the surface of the core element 7.

Furthermore, the conductor 8 comprises a second circumferential section 19 which is connected to the longitudinal section 17 at a position which is spaced form the end of the longitudinal section 17 being closest to the second end 10. Generally, the second circumferential section 19 extends transversely to the longitudinal section 17 and is arranged in this preferred embodiment perpendicular to it. Also the second circumferential section 19 extends both along the first portion 11 and the second portion 12 and is bent at an intermediate position. Thus, the second circumferential section 19 comprises two parts 19a, 19b which are inclined with respect to each other in the same manner as in case of the first circumferential section 18. The free end of the second circumferential section 19 is provided with a connecting section 20 which is arranged parallel to the longitudinal section 17 and extends along the second portion 12 to the second end 10.

A third circumferential section 21 is connected to the end of the longitudinal section 17 which is closest to the second end 10. The third circumferential section 21 is arranged perpendicular to the longitudinal section 17 and extends on the first and second portion 11, 12.

A first intermediate section 22 is connected to the free end of the third circumferential section 21. Starting from the second portion 12 of the surface of the core element 7, the first intermediate section 22 extends parallel to and across the cross sectional area of the core element 7 to its lower surface defined by bars 13 and face 14. At the lower surface, a first spring section 23 is provided which is connected to the first intermediate section 22 at its lower end and which is inclined with respect to the first intermediate section 22. Furthermore, the first spring section 23 protrudes over the lower surface. In this preferred embodiment, the first intermediate section 22 and the first spring section 23 form a first connector for the connection to a circuit board abutting on the lower surface of the core element 7. Due to the first spring section 23, the first connector is biased towards the circuit board which allows for a reliable bonding of the antenna 6 on the board without using soldering joints.

The end of the connecting section 20 closest to the second end 10 is connected to a second intermediate section 24 which is disposed in the same manner as the first intermediate section 22. Accordingly, the lower end of the second intermediate section 24 is provided with a second spring section 25 being also inclined with respect to the second intermediate section 24. In this way, the second intermediate section 24 and the second spring section 25 form a second connector which is also biased towards a circuit board.

Finally, the conductor 8 is provided with openings 26 in which the fixing pins 16 of the core element 7 engage to fix the conductor 8 in the defined position on the core element 7.

Due to the antenna design, the measuring and control device 1 has the advantage that its dimensions are kept small and that the transmission characteristics are almost direction independent. Since the portions 11, 12 of the surface of the surface of the core element 7 are inclined with respect to each other these portions 11, 12 span a volume which further improves the antenna performance.

Especially at small distances from the antenna 6 a good coverage is achieved. Furthermore, the sensitivity of the antenna 6 is neither influenced by the relative position of the antenna 6 with respect of the emitter which allows for a stable and reliable wireless connection between the elements of a blood glucose level control system.

1. Blood glucose level measuring unit comprising a housing,
an antenna being disposed within the housing and comprises:
a dielectric core element having a first end and a second end and defining a surface extending between the first end and the second end, and a conductor with:
a longitudinal section extending on the surface between the first end and the second end,
a first circumferential section which is connected to the end of the longitudinal section closest to the first end and extends transversely to the longitudinal section along the surface,
a second circumferential section which is connected to the longitudinal section spaced from the end closest to the second end and extends transversely to the longitudinal section along the surface,
a first connector connected to the end of the longitudinal section closest to the second end, and
a second connector connected to the second circumferential section.

2. Blood glucose level measuring unit according to claim 1, in which a third circumferential section connected to the end of the longitudinal section which is closest to the second end and extending transversely to the longitudinal section along the surface, wherein the first connector is connected to the free end of the third circumferential section.

3. Blood glucose level measuring unit according to claim 1, in which the surface has a first portion and a second portion wherein the first portion is inclined with respect to the second portion.

4. Blood glucose level measuring unit according to claim 3, in which the angle formed between the first portion and the second portion is between 90° and 135°.

5. Blood glucose level measuring unit according to claim 3, characterized in that the longitudinal section extends along the first portion.

6. Blood glucose level measuring unit according to claim 3, in which the first and the second circumferential sections extend along the first portion and the second portion.

7. Blood glucose level measuring unit according to claim 3, in which the third circumferential section extends along the first portion and the second portion.

8. Blood glucose level measuring unit according to claim 1, in which the first connector has a first intermediate section and the second connector has a second intermediate section and the intermediate sections extend parallel to the cross-sectional area of the core element perpendicular to the longitudinal section.

9. Blood glucose level measuring unit according to claim 8, in which the intermediate sections are arranged at the second end.

10. Blood glucose level measuring unit according to claim 8, in which a connecting section which connects the second intermediate section and the second circumferential section and which extend parallel to the longitudinal section.

11. Blood glucose level measuring unit according to claim 8, in which the core element defines a lower surface for abutting on a circuit board and that the first and the second connectors have spring sections connected to the intermediate sections, being inclined with respect to the intermediate sections and protruding over the lower surface.

12. Blood glucose level measuring unit according to claim 1, in which the core element is provided with a snap element which is adapted to lock the core element on a circuit board arranged in the housing.

13. Blood glucose level measuring unit according to claim 1, in which the core element is provided with fixing pins which protrude into openings in the conductor.
